(19) United States
(12) Patent Application Publication
(10) Pub. No.: US 2025/0143554 A1
(43) Pub. Date: May 8, 2025

(54) CAPSULE ENDOSCOPE

(71) Applicant: Ankon Medical Technologies (Shanghai) Co., LTD, Shanghai (CN)

(72) Inventors: Xiaodong DUAN, Plano, TX (US); Shaobang ZHANG, Shanghai (CN)

(73) Assignees: Ankon Medical Technologies (Shanghai) Co., LTD, Shanghai (CN); ANX IP HOLDING PTE. LTD., SG, SG (SG)

(21) Appl. No.: 18/726,111

(22) PCT Filed: Dec. 29, 2022
(86) PCT No.: PCT/CN2022/143650

§ 371 Date: Jul. 1, 2024

(30) Foreign Application Priority Data
Dec. 31, 2021 (CN) 202111673369.5

(51) Int. Cl. A61B 1/04; A61B 1/00

(52) U.S. Cl. CPC A61B 1/041; A61B 1/00064

(57) ABSTRACT

The present invention provides a capsule endoscope, including: an enclosure, including a main body portion and end portions located at both ends of the main body portion; a hardware component, arranged in an accommodating space enclosed by the enclosure, the hardware component including a battery component, the battery component being arranged in a space corresponding to the main body portion; a first thermal insulation material layer, arranged between the enclosure and the battery component; and a sealing layer, arranged between the hardware component and the thermal insulation material layer. The capsule endoscope can effectively reduce the thermal convection between the battery component and the enclosure, better concentrate the heat inside the enclosure, so that the battery component may reach a higher thermal equilibrium temperature, extend the working time of the battery component, and improve the stability of the capsule endoscope.

### FIELD OF INVENTION

The present invention relates to the field of micro medical robots, and more particularly to a capsule endoscope.

### BACKGROUND

Wireless capsule endoscope may not only capture images of the esophagus, stomach, and colon and transmit them to the outside of the body, but it can also detect areas such as the small intestine that traditional endoscopes cannot reach. Therefore, capsule endoscope has significant advantages in gastrointestinal tract examination and has received widespread attention.

However, the battery life of the capsule endoscope in working condition is a key factor limiting its ability to perform a complete examination of the gastrointestinal tract. The battery performance has a significant impact on image quality, image capture frequency, and image transmission. A complete gastrointestinal tract examination requires the battery to provide no less than 12 hours of power. Due to the physical size limitations of the capsule endoscope, it is difficult for the silver oxide or lithium battery used inside the capsule endoscope to provide the necessary power for a complete gastrointestinal examination in one go, especially if a stronger light source is used to capture higher resolution images, which requires even more battery power.

Therefore, extending the battery life to capture more images has become a pressing technical issue that needs to be solved.

### SUMMARY OF THE INVENTION

In order to technically solve above problems of the prior art, the present invention provides a capsule endoscope, comprising: an enclosure, the enclosure comprising a main body portion and end portions located at both ends of the main body portion; a hardware component, arranged in an accommodating space enclosed by the enclosure, where the hardware component comprises a battery component and the battery component is arranged in a space corresponding to the main body portion; a first thermal insulation material layer, arranged between the enclosure and the battery component; and a sealing layer, arranged between the hardware component and the first thermal insulation material layer.

The hardware component further comprises a circuit control component, and the first thermal insulation material layer is disposed between the enclosure and the circuit control component.

The thickness of the sealing layer is between 0.01 mm-0.02 mm.

The accommodating space enclosed by the enclosure is a vacuum space.

The first thermal insulation material layer comprises thermal insulation glue, aerogel material, or solid insulating material.

The first thermal insulation material layer has multiple protrusions facing the inner wall of the enclosure.

The first thermal insulation material layer is a multi-layer structure, including a first outer thermal insulation material layer close to the inner wall of the enclosure and a first inner thermal insulation material layer close to the outer periphery of the battery component. The hardness of the first outer thermal insulation material layer is less than the hardness of the first inner thermal insulation material layer.

The first thermal insulation material layer comprises a first battery thermal insulation material layer and a first circuit thermal insulation material layer. The first battery thermal insulation material layer is disposed between the enclosure and the battery component, the first circuit thermal insulation material layer is disposed between the enclosure and the circuit control component, and the thickness of the first battery thermal insulation material layer is greater than the thickness of the first circuit thermal insulation material layer.

As previously described, the capsule endoscope further comprises an image acquisition component and a second thermal insulation material layer. The image acquisition component is disposed in a space corresponding to one end portion, the second thermal insulation material layer is disposed between the image acquisition component and the enclosure, and the second thermal insulation material layer is a transparent thermal insulation material layer.

The transparent thermal insulation material layer comprises metal organic ester aerogel.

According to the present invention, the capsule endoscope can effectively reduce the thermal convection between the battery and the enclosure, better concentrate the heat inside the enclosure of the capsule endoscope, so that the battery may reach a higher thermal equilibrium temperature, extend the working time of the battery, and improve the stability of the capsule endoscope.

### BRIEF DESCRIPTION OF THE DRAWINGS

The accompanying drawings, which are included to provide a further understanding of the present invention and are incorporated in and constitute a part of the present invention, illustrate the embodiment(s) of the present invention and together with the description serve to explain the principle of the invention, but do not constitute a limitation of the present invention. In the drawings:

FIG. 1 is a structural schematic view of a capsule endoscope according to an embodiment of the present invention.

FIG. 2 is a structural schematic view of a capsule endoscope according to an embodiment of the present invention.

FIG. 3 is a structural schematic view of a sealing layer of the capsule endoscope according to an embodiment of the present invention.

FIG. 4 is a structural schematic view of a capsule endoscope according to an embodiment of the present invention.

FIG. 5 is a structural schematic view of a sealing layer of the capsule endoscope according to an embodiment of the present invention.

FIG. 6 shows a relationship curve between the battery voltage and the number of images of captured by the capsule endoscope according to an embodiment of the present invention.

FIG. 7 is a schematic view of an assembly of a capsule endoscope according to an embodiment of the present invention.

FIG. 8 is a structural schematic view of a capsule endoscope according to an embodiment of the present invention.

FIG. 9 is a structural schematic view of a capsule endoscope according to an embodiment of the present invention.

FIG. 10 is a structural schematic view of a capsule endoscope according to an embodiment of the present invention.

FIG. 11 is a structural schematic view of a capsule endoscope according to an embodiment of the present invention.

### DETAILED DESCRIPTION

To more clearly understand the objects, features, and advantages of the present invention, a detailed description is provided below in conjunction with the accompanying drawings and specific embodiments. It should be noted that, where there is no conflict, the embodiments and features described in the present invention can be combined with each other.

Many specific details are set forth in the following description to provide a thorough understanding of the present invention. However, the present invention can be practiced in other ways than those specifically described herein. Therefore, the scope of the present invention is not limited by the specific embodiments disclosed below.

### Embodiments

### (1) Embodiment 1

As shown in FIG. 1, the embodiment provides a capsule endoscope, comprising: an enclosure 1, the enclosure 1 comprising a main body portion 1-1, end portion 1-2 and end portion 1-3 located at both ends of the main body portion. The enclosure 1 encloses a space for accommodating a hardware component of the capsule endoscope, the hardware component comprises a battery component 2, and the battery component is disposed in the space corresponding to the main body portion 1-1. The hardware component may also comprise an image acquisition component, a circuit control component, a magnet, a radio frequency (abbreviated as RF) transmission component, etc. The RF transmission component is used for transmitting images captured by the image acquisition component to outside of the body, and comprises a radio frequency circuit and an antenna.

The capsule endoscope in this embodiment further comprises a first thermal insulation material layer 4 disposed between the enclosure 1 and the battery component 2.

The battery component in the capsule endoscope is generally one or more silver oxide batteries. Temperature has a significant impact on the working time of the capsule endoscope, mainly because temperature may accelerate the internal chemical reactions of the silver oxide batteries, ensuring smooth electron migration.

However, since the enclosure of the capsule endoscope is primarily made of biocompatible polymer materials, which do not generate heat themselves, the heat generated by the battery component is dissipated through air convection to the low-temperature enclosure, preventing the accumulation of heat in the battery component and causing the temperature to remain stable. The final equilibrium temperature of the battery component is low in working condition.

To reduce the heat conduction between the battery component and the enclosure of the capsule endoscope, reduce convection, and decrease the heat radiation of the battery component, the first thermal insulation material layer 4 is disposed between the enclosure 1 and the battery component 2.

Setting an thermal insulation material layer between the enclosure and the battery component may effectively reduce the heat convection between the battery component and the enclosure, ensuring that the battery component may reach a higher thermal equilibrium temperature.

The first thermal insulation layer 4 may be insulating glue, aerogel, etc. with poor thermal conductivity, or a solid insulating material with poor thermal conductivity. For example, the first thermal insulation layer may comprise thermal insulation glue in a gel state, such as nanostructured aerogel, or may comprise or consist of one or more of solid glass fibers, mineral wool insulation fibers, calcium silicate, alumina fibers, cellulose thermal insulation element, polystyrene thermal insulation element, and/or polyurethane foam thermal insulation element.

In this embodiment, the capsule endoscope also comprises a sealing layer 3 disposed between the first thermal insulation material layer 4 and the battery component 2.

To better protect the hardware components such as the battery component and ensure the effectiveness of the thermal insulation material layer, the sealing layer 3 is disposed between the battery component and the thermal insulation material layer. The battery component is sealed with a sealing layer with a thickness of 0.01 mm-0.02 mm to ensure that the battery component does not directly contact the thermal insulation glue, thus ensuring the effectiveness of the thermal insulation glue.

Especially in a case where the first thermal insulation layer is a gel-state thermal insulation glue. If the thermal insulation glue comes into contact with hardware such as the battery component, the battery component will generate heat during operation. In the situation of heat generation, the thermal insulation glue remains in a non-cured gel state. Direct contact with the battery component will affect the usage of the battery component, affecting the stability of using the capsule endoscope.

Using a sealing layer to separate the thermal insulation material layer from the battery component may better maintain the stability of using the capsule endoscope. When the first thermal insulation material layer is a solid insulating material, the sealing layer may also prevent dust and other particles from affecting the battery in the thermal insulation material layer, thus improving the operating stability of the battery component.

The material of the sealing layer 3 may be polyethylene, polypropylene, polyvinyl chloride, or any other polymer materials. The present invention is not limited to this.

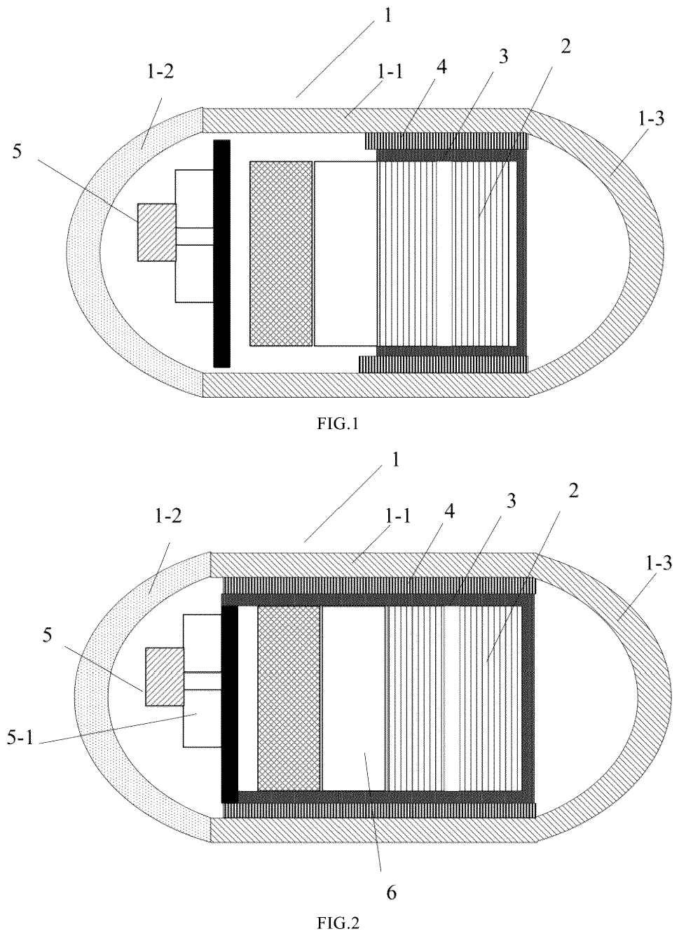

In the capsule endoscope shown in FIG. 1, the first thermal insulation material layer 4 is only disposed between the battery component 2 and the enclosure. In an embodiment, as shown in FIG. 2, in addition to the battery component 2, the hardware component also comprises a circuit control component 6. The first thermal insulation material layer may be disposed on the inner side of the entire main body portion 1-1. In addition to setting the first thermal insulation material layer between the battery component 2 and the enclosure 1, the first thermal insulation material layer 4 is also disposed between the circuit control component 6 and the enclosure 1.

Setting the first thermal insulation material layer 4 between the circuit control component 6 and the enclosure 1 may better reduce the heat conduction between the battery component and the enclosure, reduce convection, reduce battery heat radiation, and also better concentrate the heat generated by the circuit control component 6 in the working state inside the enclosure of the capsule endoscope, so that the battery component may reach a higher thermal equilibrium temperature and extend the working time of the battery component.

The capsule endoscope further comprises an image acquisition component 5, which is disposed at the end portion 1-2 of the enclosure. The capsule endoscope further comprises a RF transmission component (not shown in the figures) disposed at the other end portion 1-3 of the enclosure.

The image acquisition component 5 may comprise a lighting source and a camera, and the lighting source can be light emitting diodes (abbreviated as LED). In order to facilitate the capture of clear images, the end portion 1-2 of the enclosure corresponding to the location of image acquisition component 5 is made of a transparent material, while the main body portion 1-1 of the enclosure and the other end portion 1-3 with RF transmission component are made of non-transparent materials, but the present invention is not limited to this.

When the material of the first thermal insulation material layer 4 is a solid insulating material, the thermal insulation material layer may be fixed on the inner side of the enclosure 1 by attaching or other methods, as shown in FIG. 2.

In the case where the material in the first thermal insulation material layer 4 is a gel-state thermal insulation glue, the following method may be used to form it.

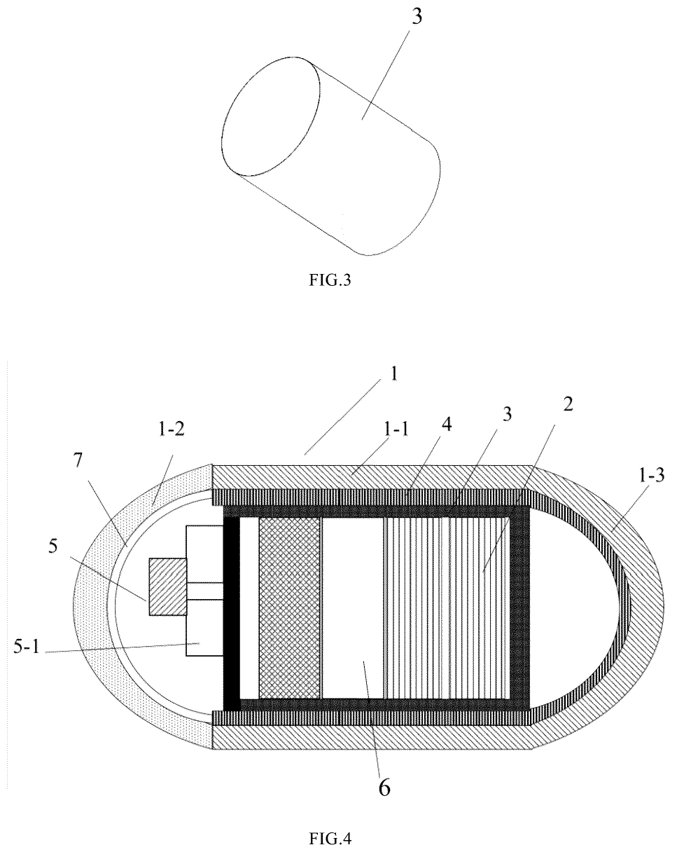

First, the hardware components of the capsule endoscope such as the battery component 2 and the circuit control component 6 are encapsulated with sealing layer 3. The schematic diagram of the structure of the sealing layer 3 (side view) is shown in FIG. 3.

Thermal insulation glue is then injected into between the hardware component wrapped in a sealing film and the enclosure 1. In one embodiment, thermal insulation glue is first added into the enclosure, then the hardware component wrapped in a sealing layer 3 is placed into the bottom of the enclosure filled with glue, and the hardware component squeezes the thermal insulation glue from the bottom to the outside of the sealing layer. In another embodiment, the hardware component wrapped in the sealing layer 3 is first placed into the enclosure 1, and then thermal insulation glue is injected into the gap between the sealing layer 3 and the enclosure 1 to a specified height. Specifically, the injected thermal insulation glue is flush with a support plate 5-1 of LED of the image acquisition component 5.

In this way, the thermal insulation glue is added between the enclosure of the capsule endoscope and the battery component, and between other hardware components in the capsule endoscope, and the thermal insulation glue will slowly fill the gaps between the enclosure and the hardware components.

The thermal insulation glue fills the interior voids of the capsule endoscope, expelling the air in the voids, which may weaken the convection between the battery component and the enclosure. The heat of the battery component is concentrated, thus achieving a long working life.

The schematic diagram of the capsule endoscope obtained in this way is shown in FIG. 4, and the first thermal insulation material layer 4 is also formed on the inner wall of the enclosure end portion 1-3, so as to improve the effect of heat accumulation of the battery component. The RF transmission component is also disposed in the space corresponding to the enclosure end portion 1-3 (not shown in the figures). The first thermal insulation material layer 4 may not be formed on the inner wall of the end portion 1-3 of the enclosure. The present invention is not limited to this.

The present invention is not limited to the above methods, and may also use the method of applying thermal insulation glue on the inner surface of the enclosure, and then placing the hardware component wrapped with the sealing layer 3.

As shown in FIG. 5, the sealing layer 3 may also be a multi-layer structure, comprising an innermost layer 3-1 and an outermost layer 3-2. The sealing layer is wound around the hardware structure of the capsule endoscope, such as the battery component, the circuit control component, and the image acquisition component, to ensure the self-insulation of the sealed capsule endoscope hardware structure, maintain the thermal equilibrium temperature for the operation of the battery component, and extend the working time of the battery component.

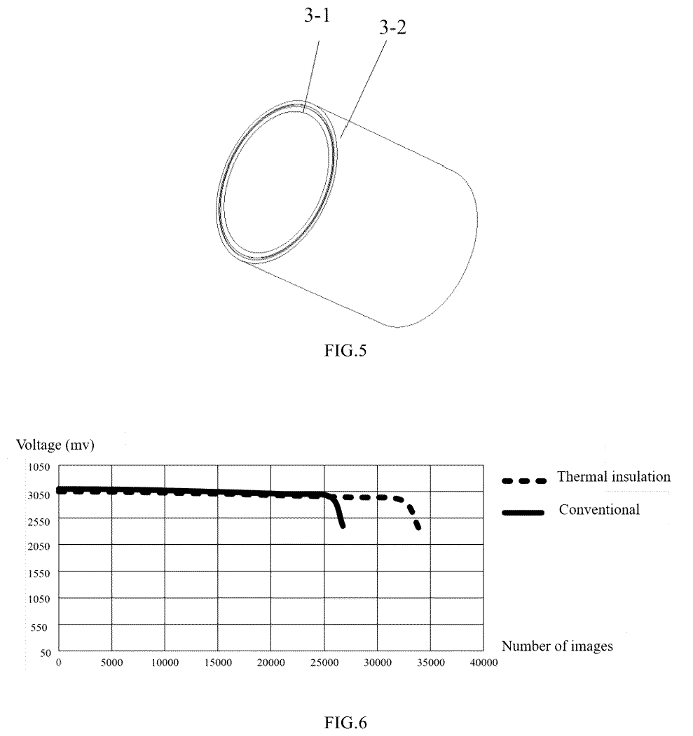

FIG. 6 specifically shows the relationship curve between the battery voltage of the capsule endoscope treated with thermal insulation material layer and the number of captured images. Under the same test conditions, the voltage drop of the capsule endoscope with the thermal insulation material layer is slower, and the number of images captured and the capture time are significantly increased compared to the untreated capsule endoscope. Through experiments, it was found that the number of images captured by the conventional capsule endoscope was 26,044, while the number of images captured by the thermal insulation-treated capsule endoscope was 34,409, an increase of 32% in the number of images captured.

Additionally, in the experiment, it can be seen that the thermal insulation treatment makes the voltage drop of the battery component of the capsule endoscope slower and steadier, indicating that the chemical reaction of the thermal insulation-treated battery is more stable.

The enclosure 1 of the capsule endoscope comprises a main body portion enclosure 1-1 and two end portion enclosures 1-2 and 1-3, wherein the end portion 1-2 is referred to as the front end portion enclosure and the end portion 1-3 as the rear end portion enclosure. In one embodiment, the main body portion enclosure 1-1 and the rear end portion 1-3 form an integral structure. After the hardware structure and the sealing layer and thermal insulation material layer are disposed in the main body portion 1-1 and the rear end portion 1-3, the final assembly stage of the front end portion enclosure and the main body portion requires completion in a vacuum operation box. Specifically, in the vacuum operation box, the gas inside the enclosure of the capsule endoscope is evacuated, and the front end portion enclosure 1-2 is glued to the main body portion enclosure 1-1 and rear end portion enclosure 1-3 using adhesive to create an accommodating space enclosed by the enclosure 1 of the capsule endoscope, which is a vacuum space, thus reducing heat convection between the battery component and the enclosure.

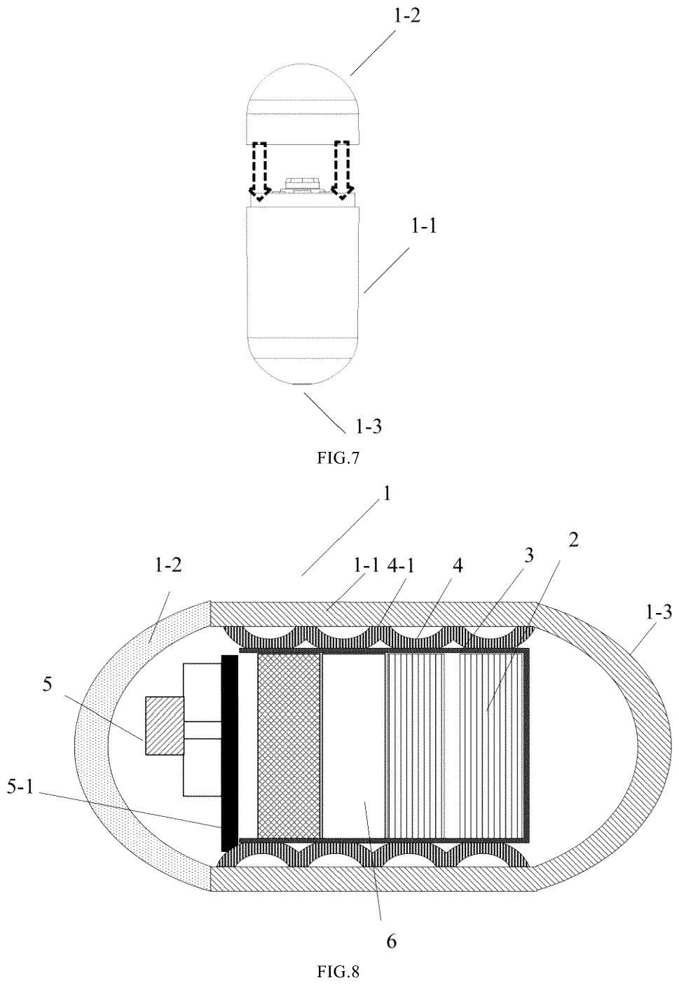

The specific assembly diagram is shown in FIG. 7. For clarity, the hardware structure and sealing layer, thermal insulation material layer, etc. in the main body portion 1-1 and the rear end portion enclosure 1-3 are omitted here.

According to the capsule endoscope provided in this embodiment, an thermal insulation material layer is disposed between the enclosure and the hardware component, which may reduce the heat conduction between the battery component and the enclosure of the capsule endoscope, concentrate the heat generated by the battery component and the circuit control component inside the enclosure of the capsule endoscope, so that the battery component may reach a higher thermal equilibrium temperature, and extend the working time of the battery.

### (2) Embodiment 2

As shown in FIG. 8, the embodiment provides another capsule endoscope, comprising: an enclosure 1, the enclosure 1 comprising a main body portion 1-1 and end portion 1-2 and end portion 1-3 located at both ends of the main body portion. The enclosure 1 encloses a space for accommodating the hardware component of the capsule endoscope, the hardware component comprises a battery component 2 and a circuit control component 6, and the battery component 2 is disposed in the space corresponding to the main body portion 1-1. The capsule endoscope further comprises a first thermal insulation material layer 4 disposed between the enclosure 1 and the battery component 2 and the circuit control component 6, and a sealing layer 3 disposed between the first thermal insulation material layer 4 and the battery component 2 and the circuit control component 6.

In this embodiment, the material, structure, and fabrication method of the first thermal insulation material layer 4 and the sealing layer 3 may be the same as in the Embodiment 1. The difference is that the first thermal insulation material layer 4 provided in this embodiment is not a flat structure, but has a plurality of protrusions 4-1 facing the inner wall of the enclosure.

The plurality of protrusions 4-1 arranged on the first thermal insulation material layer 4 can reduce the contact area between the first thermal insulation material layer 4 and the enclosure 1 of the capsule endoscope, reduce the heat conduction efficiency, further weaken the heat conduction between the battery component and the enclosure of the capsule endoscope, and concentrate the heat inside the enclosure of the capsule endoscope.

### (3) Embodiment 3

As shown in FIG. 9, the embodiment provides another capsule endoscope, comprising: an enclosure 1, the enclosure 1 comprising a main body portion 1-1 and end portion 1-2 and end portion 1-3 located at both ends of the main body portion. The enclosure 1 encloses a space for accommodating the hardware component of the capsule endoscope, the hardware component comprises a battery component 2 and a circuit control component 6, and the battery component 2 is disposed in the space corresponding to the main body portion 1-1. The capsule endoscope further comprises a first thermal insulation material layer disposed between the enclosure 1 and the battery component 2 and the circuit control component 6, and a sealing layer 3 disposed between the first thermal insulation material layer and the battery component 2 and the circuit control component 6.

The material, structure, and fabrication method of the sealing layer 3 in this embodiment may be the same as in Embodiment 1. In this embodiment, the first thermal insulation material layer is a multi-layer structure, comprising a first outer thermal insulation material layer 4-2 close to the inner wall of the enclosure and a first inner thermal insulation material layer 4-3 close to the outer periphery of the hardware component. The hardness of the first outer thermal insulation material layer 4-2 is smaller than the hardness of the first inner thermal insulation material layer 4-3.

The first inner thermal insulation material layer 4-3 may be a solid thermal insulation material layer, or may comprise or consist of one or more of solid glass fiber, mineral wool insulation fiber, calcium silicate, alumina fiber, cellulose thermal insulation element, polystyrene thermal insulation element, and/or polyurethane foam thermal insulation element. The solid thermal insulation material layer can be more stably fixed inside the enclosure of the capsule endoscope, reducing heat conduction between the battery component and the enclosure while ensuring the stable operation of the battery component and the circuit control component, and reducing vibrations of the battery component and the circuit control component.

The first outer thermal insulation material layer 4-2 may be a flexible material or a thermal insulation material in a gel state with hardness lower than the first inner thermal insulation material layer 4-3. The first outer thermal insulation material layer 4-2 with lower hardness is in contact with the enclosure of the capsule endoscope, does not increase the overall hardness of the capsule endoscope, and does not compromise the biocompatibility of the capsule endoscope with the human body.

In addition, multiple thermal insulation material layers may better reduce thermal conduction efficiency, allowing heat to be better concentrated inside the enclosure of the capsule endoscope.

### (4) Embodiment 4

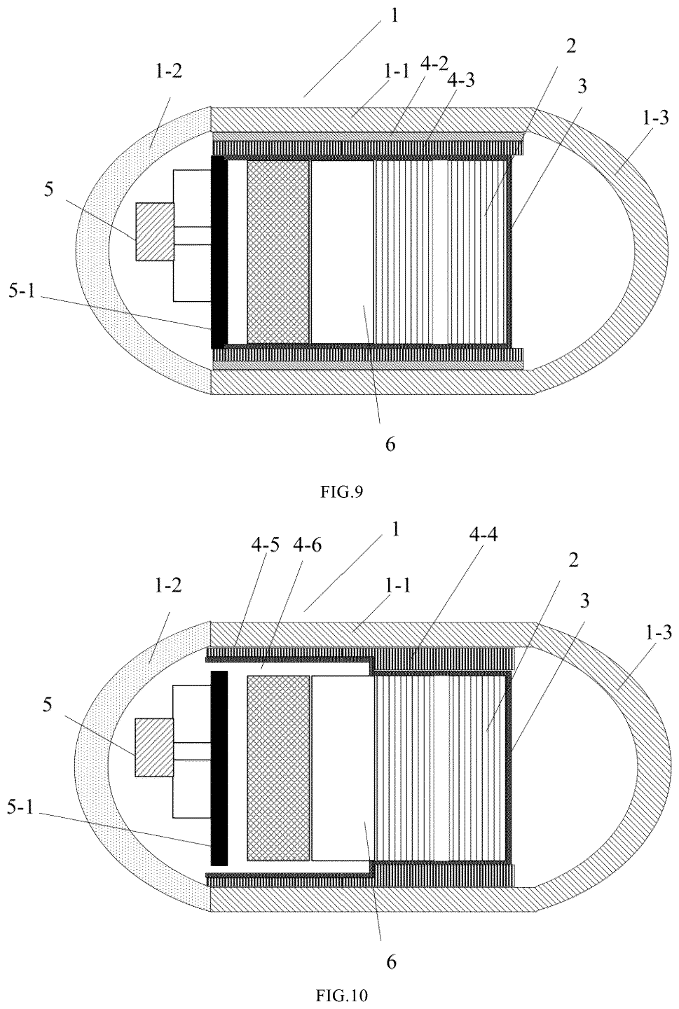

As shown in FIG. 10, the embodiment provides another capsule endoscope, comprising: an enclosure 1, the enclosure 1 comprising a main body portion 1-1 and end portion 1-2 and end portion 1-3 located at both ends of the main body portion. The enclosure 1 encloses a space for accommodating the hardware component of the capsule endoscope, the hardware component comprises a battery component 2 and a circuit control component 6, and the battery component 2 is disposed in the space corresponding to the main body portion 1-1. The capsule endoscope further comprises a first thermal insulation material layer disposed between the enclosure 1 and the battery component 2 and the circuit control component 6, and a sealing layer 3 disposed between the first thermal insulation material layer and the battery component 2 and the circuit control component 6.

The material, structure, and fabrication method of the sealing layer 3 in this embodiment may be the same as in Embodiment 1. The material and fabrication method of the first thermal insulation material layer in the embodiment may also be the same as in Embodiment 1. However, the structure of the first thermal insulation material layer in this embodiment is different from that of Embodiment 1.

In this embodiment, the first thermal insulation material layer comprises a first battery thermal insulation material layer 4-4 and a first circuit thermal insulation material layer 4-5. The first battery thermal insulation material layer 4-4 is disposed between the enclosure and the battery component 2, and the first circuit thermal insulation material layer 4-5 is disposed between the enclosure 1 and the circuit control component 6. The thickness of the first battery thermal insulation material layer 4-4 is greater than the thickness of the first circuit thermal insulation material layer 4-5.

As the sealing layer 3 is made of flexible material, the thickness of the first circuit thermal insulation material layer 4-5 disposed between the circuit control component and the enclosure is relatively small. The sealing layer 3 is adhered to the first circuit thermal insulation material layer 4-5, leaving a certain amount of reserved space 4-6 between the circuit control component and the enclosure.

The circuit control component 6 comprises a printed circuit board (abbreviated as PCB) and various traces. With the current trend of miniaturization of capsule endoscopes, the space left for various traces is relatively small, which may easily lead to broken traces. Setting the thickness of the first circuit thermal insulation material layer 4-5 to be smaller than the thickness of the first battery thermal insulation material layer 4-4 allows for a certain amount of reserved space 4-6 for the circuit control component, preventing excessive occupation of space by the thermal insulation material layer and ensuring that the circuit wiring is not squeezed by the inner wall of the enclosure, thus avoiding the occurrence of fractures or breakages. This setup can guarantee the service life of the capsule endoscope while also taking advantage of the heat accumulation within the capsule enclosure.

### (5) Embodiment 5

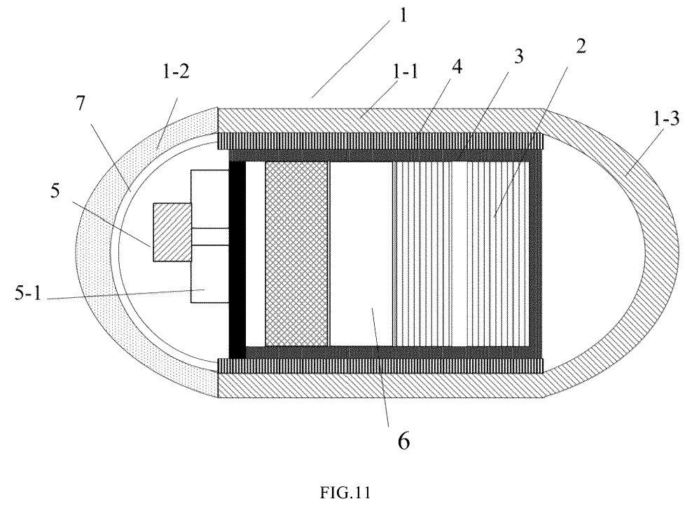

As shown in FIG. 11, the embodiment provides another capsule endoscope, comprising: an enclosure 1, the enclosure 1 comprising a main body portion 1-1 and end portion 1-2 and end portions 1-3 located at both ends of the main body portion. The enclosure 1 encloses a space for accommodating the hardware component of the capsule endoscope, the hardware component comprises a battery component 2 and a circuit control component 6, and the battery component 2 is disposed in the space corresponding to the main body portion 1-1. The capsule endoscope further comprises a first thermal insulation material layer disposed between the enclosure 1 and the battery component 2 and the circuit control component 6, and a sealing layer 3 disposed between the first thermal insulation material layer and the battery component 2 and the circuit control component 6.

The capsule endoscope further comprises an image acquisition component 5 and a second thermal insulation material layer 7 disposed at the end portion 1-2 of the enclosure 1. The image acquisition component is disposed in a space corresponding to the end portion, and the second thermal insulation material layer 7 is disposed between the image acquisition component 5 and the enclosure 1, and the second thermal insulation material layer is a transparent thermal insulation material layer.

The image acquisition component 5 comprises a lighting source and a camera. The image acquisition component needs to capture clear images of the human body, so the end portion 1-2 of the corresponding enclosure 1 of the image acquisition component 5 is usually made of transparent material. The image acquisition component 5 will emit heat during operation, which will be dissipated to the outside through the enclosure 1.

A second thermal insulation material layer 7 is disposed between the image acquisition component and the enclosure to reduce heat convection and radiation, concentrating heat inside the enclosure of the capsule endoscope, but without reducing the light transmittance of the end portion 1-2 of the enclosure 1, which would affect the image capture of the camera. Therefore, a transparent thermal insulation material is chosen to form the second thermal insulation material layer 7. In one embodiment, a transparent thermal insulation material comprising metal organic ester aerogel is selected. In one embodiment, the second thermal insulation material layer 7 may be coated on the inner wall of the end portion 1-2 of the enclosure. However, the present invention is not limited to this.

Aerogel materials prepared with metal organic esters have a transparency of 90%, a porosity of 95%, a thermal conductivity of 0.015 W/mK-0.020 W/mK, good thermal insulation properties, and high transparency. They can reduce the heat generated by the image acquisition component from diffusing to the outside of the enclosure, while also taking into account the quality of the photographs.

When preparing the capsule endoscope, transparent thermal insulation material may be coated on the inner wall of the end portion 1-2 of the enclosure first, and then the end portion 1-2 of the enclosure may be assembled with the main body portion and the other end portion in a vacuum state, but the present invention is not limited to this.

According to this embodiment, the capsule endoscope can better concentrate the heat generated by the hardware components inside the enclosure of the capsule endoscope, which is beneficial for maintaining the thermal equilibrium temperature for the battery to extend the working time of the battery, while also considering image capture.

In the present invention, the terms “first” and “second” are only used for descriptive purposes and should not be understood as indicating or implying relative importance; the term “multiple” means two or more. For those skilled in the art, the specific meanings of the terms in the present invention may be understood on a case-by-case basis.

The above descriptions are only preferred embodiments of the present invention, and are not intended to limit the present invention. For those skilled in the art, the present invention may have various modifications and changes. Any modifications, equivalent replacements, improvements, etc. made within the spirit and principles of the present invention shall be included within the protection scope of the present invention.

The listing of the claims:

1. A capsule endoscope, comprising:
an enclosure, comprising a main body portion and end portions located at both ends of the main body portion;
a hardware component disposed within an accommodating space enclosed by the enclosure, wherein the hardware component comprises a battery component, the battery component is disposed in a space corresponding to the main body portion;
a first thermal insulation material layer disposed between the enclosure and the battery component; and
a sealing layer disposed between the hardware component and the first thermal insulation material layer.

2. The capsule endoscope of claim 1, wherein the hardware component further comprises a circuit control component, and the first thermal insulation material layer is disposed between the enclosure and the circuit control component.

3. The capsule endoscope of claim 1, wherein the thickness of the sealing layer is between 0.01 mm-0.02 mm.

4. The capsule endoscope of claim 1, wherein the accommodating space enclosed by the enclosure is a vacuum space.

5. The capsule endoscope of claim 1, wherein the first thermal insulation material layer comprises insulating glue, aerogel material, or solid insulating material.

6. The capsule endoscope of claim 1, wherein the first thermal insulation material layer has multiple protrusions facing the inner wall of the enclosure.

7. The capsule endoscope of claim 1, wherein the first thermal insulation material layer is a multi-layer structure, comprising a first outer thermal insulation material layer close to the inner wall of the enclosure and a first inner thermal insulation material layer close to the outer periphery of the battery component, wherein the hardness of the first outer thermal insulation material layer is less than the hardness of the first inner thermal insulation material layer.

8. The capsule endoscope of claim 2, wherein the first thermal insulation material layer comprises a first battery thermal insulation material layer and a first circuit thermal insulation material layer, wherein the first battery thermal insulation material layer is disposed between the enclosure and the battery component, the first circuit thermal insulation material layer is disposed between the enclosure and the circuit control component, and the thickness of the first battery thermal insulation material layer is greater than the thickness of the first circuit thermal insulation material layer.

9. The capsule endoscope of claim 1, further comprising an image acquisition component and a second thermal insulation material layer, wherein the image acquisition component is disposed in a space corresponding to one end portion, the second thermal insulation material layer is disposed between the image acquisition component and the enclosure, and the second thermal insulation material layer is a transparent thermal insulation material layer.

10. The capsule endoscope of claim 9, wherein the second thermal insulation material layer is disposed on the inner wall at the end portion of the main body portion, and the transparent thermal insulation material layer comprises metal organic ester aerogel.
